(19) United States
(12) Patent Application Publication
(10) Pub. No.: US 2026/0368896 A1
(43) Pub. Date: Nov. 19, 2026

(54) THREE-DIMENSIONAL ORGAN MEDICAL IMAGE FEATURE CAPTURING SYSTEM AND METHOD THEREOF FOR REDUCING FALSE POSITIVES

(71) Applicant: Findings Tech Co., Ltd., Taipei (TW)

(72) Inventors: CHUN-PENG HSU, Taipei (TW); Mao-Yuan HSU, Taipei (TW); JIA-MIN LIN, Taipei (TW); Dong-You Jhong, Taipei (TW); SZU-AN CHEN, Taipei (TW); Lu Liu, Taipei (TW); Yu-Chen Shih, Taipei (TW)

(21) Appl. No.: 19/209,621

(22) Filed: May 15, 2025

(51) Int. Cl. G06T 7/00; A61B 5/00; A61B 5/055; G01R 33/56

(52) U.S. Cl. CPC G06T 7/0014; A61B 5/055; A61B 5/7267; G01R 33/5608; G06T 2207/10088; G06T 2207/20081; G06T 2207/30056

(57) ABSTRACT

Three-dimensional organ medical image feature capturing system and method for classifying organ and false positives, organ cross-sectional image layers of a same type of three-dimensional organs are obtained by magnetic resonance imaging, a specific organ marking image is generated by an AI analysis system for the layers, the layer with the image is imported to an image analysis unit, a center of gravity point is generated for the image, the layer with the point and image is imported to a feature capture unit for calculating distance data based on the point and a reference point, calculating area data based on the image, generating a layer number data set based on total layer number, current layer number and image code information, importing the distance, area data and set to a feature operation unit and generating a matching result, a post-processing unit deletes or retains the image based on the result.

### BACKGROUND OF THE INVENTION

### Field of Invention

The invention relates to a feature capturing system, and more particularly to a three-dimensional organ medical image feature capturing system and a method thereof for reducing false positives.

### Related Art

Currently, in medical image processing, whether it is the results produced by neural network models or user marking, it is inevitable that the wrong area will be selected and false positives will occur. The reason is that the training of neural network models is relatively complex and requires a large amount of training costs (such as a large number of correctly marked medical images) and high-quality training data. User marking will have the problem of human marking errors. These will cause the wrong organ area to be marked on the medical image, which will in turn affect the diagnosis of the patient.

For example, magnetic resonance imaging (MRI) technology can be used to obtain a plurality of organ cross-sectional image layers of a same type of three-dimensional organs. When processing organ (liver) medical image graphics, whether it is the result produced by the neural network or the user's unintentional marking error, it is inevitable that the wrong area is selected and there will be a false positive, that is, the non-liver areas are marked.

The detailed features and advantages of the invention will be described in detail in the following embodiments. The content is sufficient to enable any person with ordinary skill in the art to understand the technical content of the invention and implement it accordingly, and in accordance with the content, the claims and the drawings disclosed in this specification, any person with ordinary skill in the art can easily understand the related objects and advantages of the invention.

### SUMMARY OF THE INVENTION

A main object of the invention is to obtain accurate organ area selection effect without requiring a very large amount of organ cross-sectional image layer information and operational costs of judgment and analysis, effectively excluding non-preset organ markings in cross-sectional image layers, thereby reducing a false positive rate and improving an accuracy rate.

In order to achieve the above-mentioned object, based on anatomical features, a feature capturing method designed based on anatomy and basic physiological characteristics is developed to serve as a classification basis for organ positives and false positives, the invention is a three-dimensional organ medical image feature capturing system for reducing false positives, a plurality of organ cross-sectional image layers of a same type of three-dimensional organs are obtained by a magnetic resonance imaging (MRI), at least one specific organ marking image is generated by an AI analysis system for the organ cross-sectional image layer of each of the organs, the system comprises: an image analysis unit for importing the organ cross-sectional image layer with the specific organ marking image, the image analysis unit generates a center of gravity point for the specific organ marking image; a feature capture unit for importing the organ cross-sectional image layer with the center of gravity point and the specific organ marking image, the feature capture unit calculates at least one distance datum based on the center of gravity point and at least one reference point, calculates at least one area datum based on the specific organ marking image, generates a layer number data set based on a total layer number information, a current layer number information and an image code information; a feature operation unit for importing the distance datum, the area datum and the layer number data set and generating a matching result; and a post-processing unit, the post-processing unit selects to delete or retain the specific organ marking image based on the matching result.

According to one embodiment of the invention, further comprising a storage unit, the storage unit storing the reference point and being used for importing the reference point to the feature capture unit.

According to one embodiment of the invention, wherein the organ cross-sectional image layer comprises the total layer number information and the current layer number information.

According to one embodiment of the invention, wherein before the system is used, the reference point must be established and stored in the storage unit, the organ cross-sectional image layers are used to mark reference truth regions, the image analysis unit is then used to generate reference center of gravity points of the reference truth regions, and the reference point is obtained by performing an average calculation on the reference center of gravity points.

In order to achieve the above-mentioned object, the invention is a three-dimensional organ medical image feature capturing method for reducing false positives, obtaining a plurality of organ cross-sectional image layers of a same type of three-dimensional organs by a magnetic resonance imaging (MRI), generating at least one specific organ marking image by an AI analysis system for the organ cross-sectional image layer of each of the organs, comprising:
a. importing the organ cross-sectional image layer with the specific organ marking image to an image analysis unit;
b. analyzing the specific organ marking image and generating a center of gravity point;
c. importing the organ cross-sectional image layer with the center of gravity point and the specific organ marking image to a feature capture unit;
d. calculating at least one distance datum based on the center of gravity point and at least one reference point, calculating at least one area datum based on the specific organ marking image, generating a layer number data set based on a total layer number information, a current layer number information and an image code information;
e. importing the distance datum, the area datum and the layer number data set to a feature operation unit, the feature operation unit generating a matching result; and f. importing the matching result to a post-processing unit, the post-processing unit selecting to delete or retain the specific organ marking image based on the matching result.

According to one embodiment of the invention, wherein the reference point is imported to the feature capture unit from a storage unit.

According to one embodiment of the invention, wherein the organ cross-sectional image layer comprises the total layer number information and the current layer number information.

According to one embodiment of the invention, wherein before the system is used, the reference point must be established and stored in the storage unit, the organ cross-sectional image layers are used to mark reference truth regions, the image analysis unit is then used to generate reference center of gravity points of the reference truth regions, and the reference point is obtained by performing an average calculation on the reference center of gravity points.

### BRIEF DESCRIPTION OF THE DRAWINGS

FIG. 1 is a schematic diagram of a three-dimensional organ medical image feature capturing system for reducing false positives according to the invention.

FIG. 2 is a schematic diagram of organ cross-sectional image layers and specific organ marking images according to the invention.

FIG. 3 is a schematic diagram of data processing of the three-dimensional organ medical image feature capturing system for reducing false positives according to the invention.

FIG. 4 is a schematic diagram of center of gravity points according to the invention.

FIG. 5 is a schematic diagram of the organ cross-sectional image layers, reference truth regions, and reference center of gravity points according to the invention.

FIG. 6 is a schematic diagram showing that a feature capture unit of the invention calculates a distance datum based on one of the center of gravity points and reference points.

FIG. 7 is a schematic diagram of generating regions of area data according to the invention.

FIG. 8 is a schematic diagram of a layer number data set according to the invention.

FIG. 9 is a schematic diagram of the layer number data sets, the distance data, the area data, and matching results according to the invention.

FIG. 10 is a schematic diagram of deleting false positive marking images according to the invention.

FIG. 11 is a flow chart of a three-dimensional organ medical image feature capturing method for reducing false positives according to the invention.

### DETAILED DESCRIPTION OF THE INVENTION

The following specific embodiments illustrate the implementation mode of the invention. Any person with ordinary skill in the art can easily understand the other advantages and efficacies of the invention from the content disclosed in this specification.

The structures, proportions and sizes depicted in the accompanied drawings in this specification are only used to match with the content disclosed in the specification for understanding and reading by those skilled in the art, and are not intended to limit the conditions that can be implemented by the invention, and therefore are not technically significant. Any structural modifications, changes in proportional relationship or adjustment of sizes without affecting the efficacies generated and the objects achieved by the invention, should still fall within the scope covered by the technical content disclosed by the invention. At the same time, the terms such as “one”, “two”, “above”, etc. cited in this specification are only for clarity of description, and are not used to limit the scope of the invention, changes or adjustments in their relative relationships, without substantial changes to the technical content, should also be regarded as the scope of the invention that can be implemented.

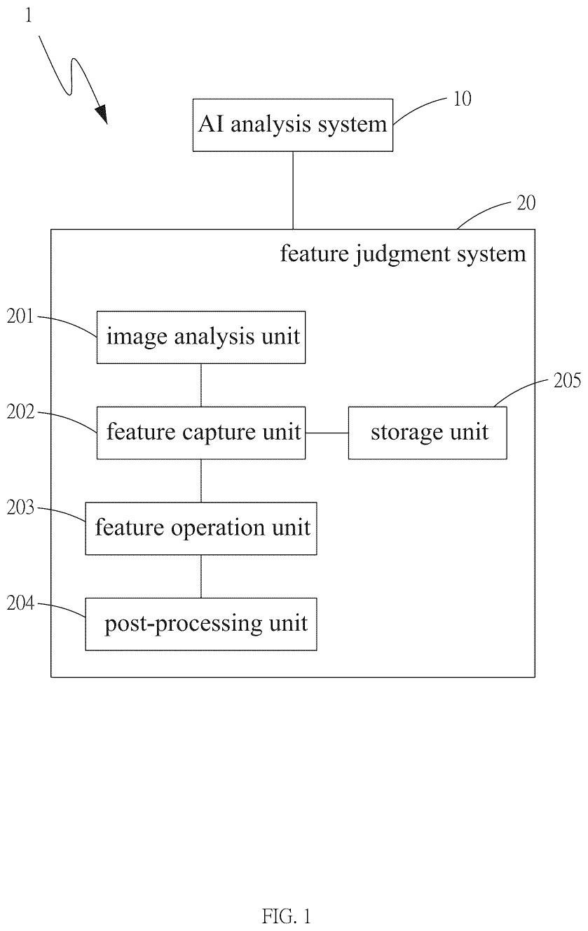

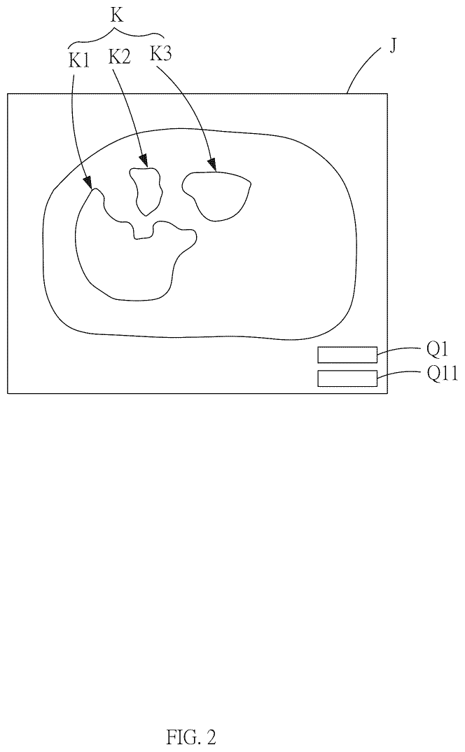

Please refer to FIGS. 1 and 2. The invention is a three-dimensional organ medical image feature capturing system 1 for reducing false positives. A multi-layer cross-sectional image of a three-dimensional organ is obtained by a magnetic resonance imaging (MRI), that is, a “slice-type” two-dimensional image of an organ. If the organ is a liver, for example, a plurality of organ cross-sectional image layers J (for example, 50 layers) are generated for the liver organ, and one of the organ cross-sectional image layers J is imported to an AI analysis system 10. That is, one of the layers is selected from the 50 organ cross-sectional image layers J and imported to the AI analysis system 10. Then, taking a third layer being used as an example, the organ cross-sectional image layer J with a specific organ marking image K is generated, and then the organ cross-sectional image layer J with the specific organ marking image K is imported to a feature judgment system 20 to determine whether the specific organ marking image K is a false positive organ area, wherein the feature judgment system 20 comprises an image analysis unit 201, a feature capture unit 202, a feature operation unit 203, a post-processing unit 204, and a storage unit 205, wherein the organ cross-sectional image layer J further comprises a total layer number information Q1 and a current layer number information Q11, the total layer number information Q1 is all cross-sectional layer number information of the three-dimensional organ, taking the above example, that is, 50 layers, the current layer number information Q11 is a layer number of the organ cross-sectional image layer J currently imported to the AI analysis system 10, taking the above example, that is, the third layer.

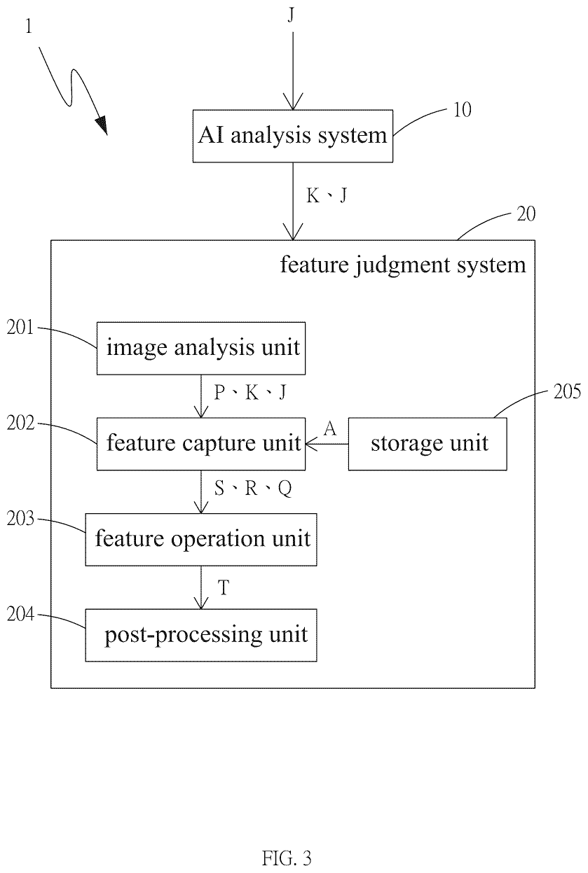

Please refer to FIGS. 2 and 3, the organ cross-sectional image layer J is imported to the AI analysis system 10, and the AI analysis system 10 generates the organ cross-sectional image layer J with the at least one specific organ marking image K, wherein the AI analysis system 10 is an artificial intelligence image recognition model, such as a residual network model (ResNet), a U-shaped network model (U-Net), or a masked region convolutional neural network model (Mask R-CNN). To further illustrate, the AI analysis system 10 is a trained artificial intelligence image recognition model, which is applied to the field of medical image analysis, and is used to mark a specific organ region in the organ cross-sectional image layer J, i.e., the specific organ marking image K. In this embodiment, there are three specific organ marking images K1, K2, K3 of the liver organ cross-sectional image layer J.

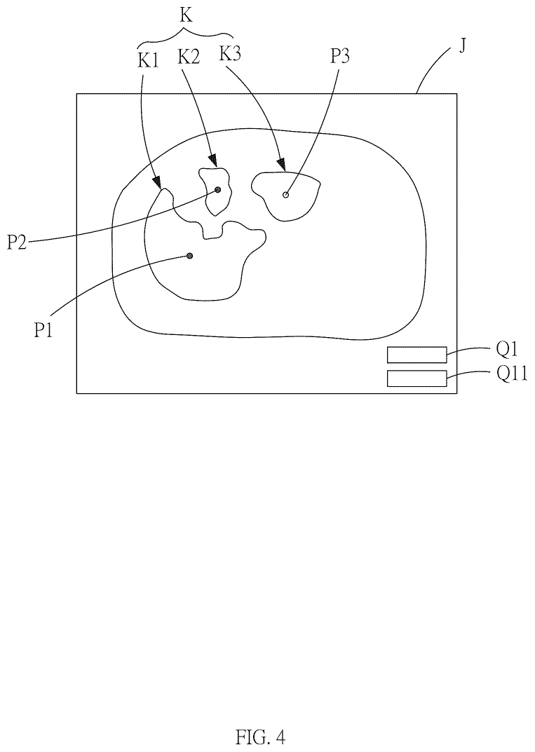

Please refer to FIGS. 3 and 4, the organ cross-sectional image layer J with the specific organ marking image K is imported to the image analysis unit 201, and the image analysis unit 201 generates a center of gravity point P for the specific organ marking image K, wherein the image analysis unit 201 can be a calculator, which is software, device or system that is capable of executing data processing, logical operation and mathematical calculation, such as: CPU, MCU, etc., especially a center of gravity calculator. In this embodiment, center of gravity points P1, P2, P3 are the center of gravity of the specific organ marking images K1, K2, K3. According to shapes of the specific organ marking images K1, K2, K3, the image analysis unit 201 has a variety of calculation methods to generate the center of gravity points P1, P2, P3, such as: weighted average of each coordinate, integration, polygon center of gravity formula, image moment operation, etc.

Please refer to FIG. 3, the organ cross-sectional image layer J, the specific organ marking image K, the center of gravity point P and at least one reference point A are imported to the feature capture unit 202, the feature capture unit 202 calculates at least one distance datum S according to the center of gravity point P and the reference point A, the feature capture unit 202 calculates at least one area datum R according to the specific organ marking image K, the feature capture unit 202 generates a layer number data set Q according to whether the total layer number information Q1 has the specific organ marking image K, wherein the feature capture unit 202 is software, device or system that is capable of executing data processing, logical operation and mathematical calculation, such as: CPU, MCU, etc. The feature capture unit 202 is used to capture feature data from the organ cross-sectional image layer J, the specific organ marking image K, the center of gravity point P and the reference point A, and the feature data comprise the at least one distance datum S, the at least one area datum R and the at least one layer number data set Q. In addition, the storage unit 205 can comprise a volatile and/or non-volatile storage device, such as random access memory (RAM), read-only memory (ROM), flash memory, hard disk drive (HDD), solid state drive (SSD). The storage unit 205 is used to store a reference point information, and the reference point information comprises the at least one reference point A of each of the organ cross-sectional image layers J.

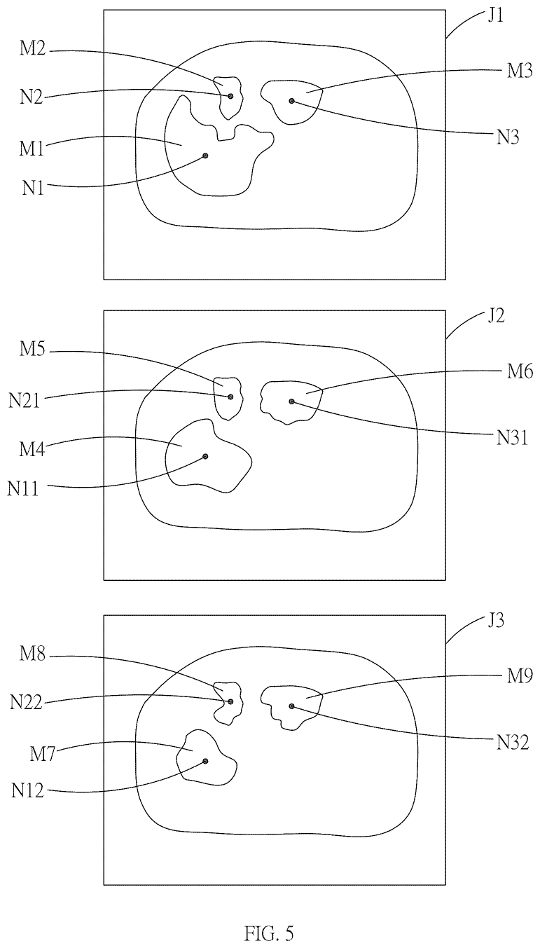

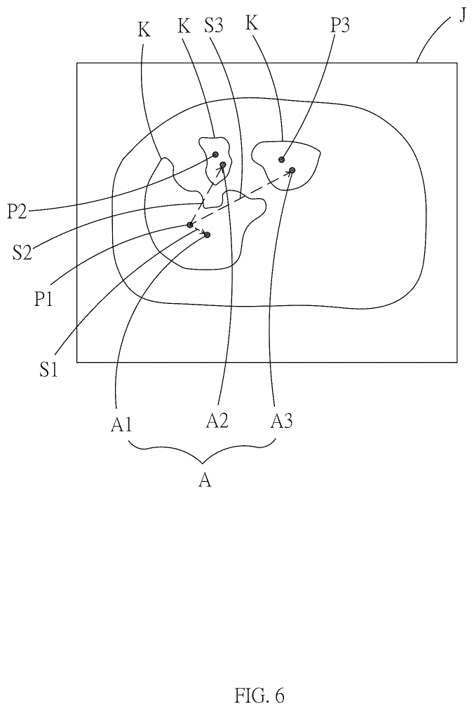

Please refer to FIGS. 5 and 6, wherein the organ cross-sectional image layer J with the center of gravity point P and the specific organ marking image K is imported to the feature capture unit 202. The feature capture unit 202 uses the center of gravity point P and reads the at least one reference point A from the storage unit 205 to calculate the at least one distance datum S. In addition, the reference point A is calculated and generated by a plurality of reference center of gravity points N. Before the system 1 is used, the reference point A must be established and stored in the storage unit 205. In this embodiment, liver is taken as an example. Firstly, reference truth regions M1~M9 (correct liver region images) in a same layer of organ cross-sectional image layers J1~3 of multiple livers are marked, that is, the reference truth regions M1~M9 in the same layer of the organ cross-sectional image layers J of the different livers are marked. An artificial intelligence image recognition model (such as the AI analysis system 10) or an expert can be used to manually mark and obtain the organ cross-sectional image layers J1-3 with the reference truth regions M1-M9, and then reference center of gravity points N1, N11, N12, N2, N21, N22, N3, N31, N32 of the reference truth regions M1-M9 are generated by the image analysis unit 201, and average calculation is performed for the reference center of gravity points N1, N11, N12, N2, N21, N22, N3, N31, N32 to obtain an average value. The average value is reference points A1, A2, A3. Further explanation, when the reference center of gravity points N1, N11, N12, N2, N21, N22, N3, N31, N32 are averaged, the average value is calculated based on the reference center of gravity points N1, N11, N12, N2, N21, N22, N3, N31, N32 of the corresponding reference truth regions M1-M9. Taking FIG. 5 as an example, N1, N11, N12 are a set, N2, N21, N22 are a set, and N3, N31, N32 are a set. An average value is calculated separately, so the three reference points A1, A2, A3 are obtained, and the reference points A1, A2, A3 are stored in the storage unit 205. In addition, the center of gravity point P and the reference point A are both coordinate data. Therefore, a distance between two coordinates can be calculated based on data of the two coordinates to obtain the distance datum S. For further explanation, please refer to FIG. 6. In this embodiment, distance data are calculated using one of the center of gravity points P1, P2, P3 and the reference point A. Since there are three reference points, the center of gravity point P1 and the reference points A1, A2, A3 are calculated to obtain three sets of distance data S1, S2, S3. Similarly, the center of gravity points P2, P3 also obtain three sets of distance data, which will not be repeated herein.

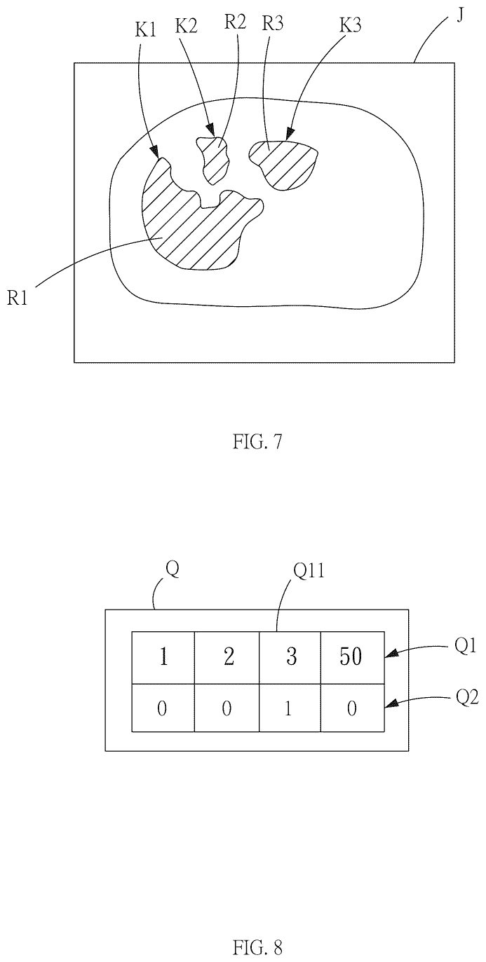

Please refer to FIG. 7, the organ cross-sectional image layer J with the center of gravity point P and the specific organ marking image K is imported to the feature capture unit 202, and the feature capture unit 202 calculates the specific organ marking image K to generate the at least one area datum R. To further illustrate, the specific organ marking images K1, K2, K3 are closed figures. The feature capture unit 202 uses mathematical formulas and programs to calculate areas of the closed figures to generate the at least one area datum R. As shown in areas with inclined lines in FIG. 7, the areas with inclined lines of the specific organ marking images K1, K2, K3 are calculated to generate area data R1, R2, R3 respectively.

As shown in FIG. 8, the organ cross-sectional image layer J with the center of gravity point P and the specific organ marking image K is imported to the feature capture unit 202. The feature capture unit 202 generates the layer number data set Q according to the total layer number information Q1, the current layer number information Q11 and an image code information Q2. To further illustrate, the total layer number information Q1 is the information of a total number of layers of the liver organ cross-sectional image layer J. For example, the liver organ cross-sectional image layer J has 50 layers, the total layer number information Q1 is 1~50, the current layer number information Q11 is a layer number to which the organ cross-sectional image layer J belongs. For example, the organ cross-sectional image layer J is a slice image of a third layer of the liver organ, and the current layer number information Q11 is 3. The image code information Q2 can be a number, data, parameter, etc., which is used to indicate a state of the specific organ marking image K of the current organ cross-sectional image layer J, that is, whether the current organ cross-sectional image layer J has the specific organ marking image K. For example, if the organ cross-sectional image layer J has the specific organ marking image K, the image code information Q2 is 1, otherwise, the image code information Q2 is 0. The image code information Q2 can be adjusted according to actual applications and is not limited to the above 1 or 0. After the feature capture unit 202 obtains the total layer number information Q1, the current layer number information Q11 and the image code information Q2, the total layer number information Q1, the current layer number information Q11 and the image code information Q2 are integrated to become the layer number data set Q. For further explanation, as shown in FIG. 8, when the layer number data set Q comprises the total layer number information Q1, the current layer number information Q11 and the image code information Q2, the total layer number information Q1 of the third layer of the liver is 1~50, the current layer number information Q11 is 3, and the third layer has the specific organ marking images K1, K2, K3, so the image code information Q2 is 1. In addition, the image code information Q2 can also comprise data of other layers according to usage requirements. For example, in FIG. 8, since the current layer does not have other layer numbers of the organ cross-sectional image layer J, the image code information Q2 of the other layer numbers is 0.

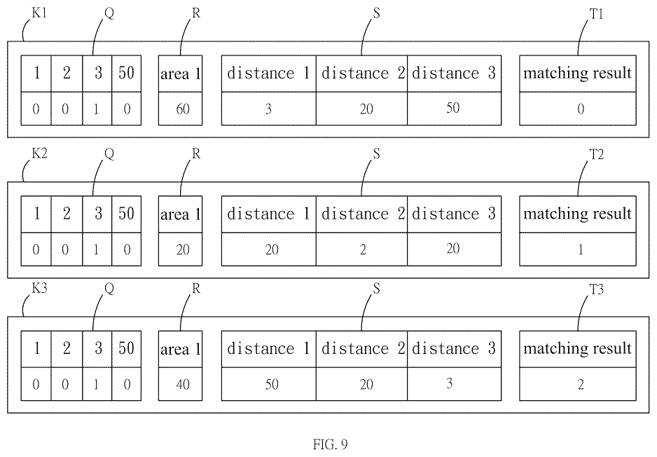

Please refer to FIG. 3, the distance data S, the area data R and the layer number data set Q are imported to the feature operation unit 203, and the feature operation unit 203 generates a matching result T, wherein the feature operation unit 203 is an AI classifier, which is a trained AI classifier that is capable of performing pattern recognition according to input data and outputting a corresponding classification result, which can be implemented by one or more neural network models (such as convolutional neural network CNN, long short-term memory network LSTM, transformer). To further illustrate feature data of the specific organ marking images K1, K2, K3: the distance data S, the area data R, and the layer number data set Q are imported to the feature operation unit 203, and the feature operation unit 203 generates the matching result T, wherein the matching result T can be a number, data, parameter, etc., which is used to indicate whether the organ cross-sectional image layer J is a correct organ area or a false positive organ area. For example, please refer to FIG. 9, which is a schematic diagram of the distance data S, the area data R, the layer number data set Q, and the matching result T of the three different specific organ marking images K1, K2, K3 of a third layer of the liver organ cross-sectional image layer J. The above-mentioned feature data are imported to the feature operation unit 203, and the feature operation unit 203 generates three sets of matching results T1, T2, T3, which are shown as numbers 0, 1, 2 in FIG. 9. In addition, the feature operation unit 203 must be trained before use. The feature data used are the distance data S, the area data R and the layer number data set Q obtained from a correct organ region of the organ cross-sectional image layer J after confirmation. The corresponding result data (i.e., the matching result T) is a number, data, parameter, etc. to represent correct data. Conversely, if the distance data S, the area data R and the layer number data set Q are obtained from a false-positive organ region of the organ cross-sectional image layer J after confirmation, result data corresponding to the matching result T represent false-positive data; if the result data are set to 0, 1 as correct data and 2 as false-positive data, referring to FIG. 9, a matching result 2 is false-positive data, and the corresponding specific organ marking image K3 is a false-positive region. In addition, since the specific organ marking images K of each layer are different, the matching result T or result data can be adjusted according to a quantity of the specific organ marking images K of a current layer.

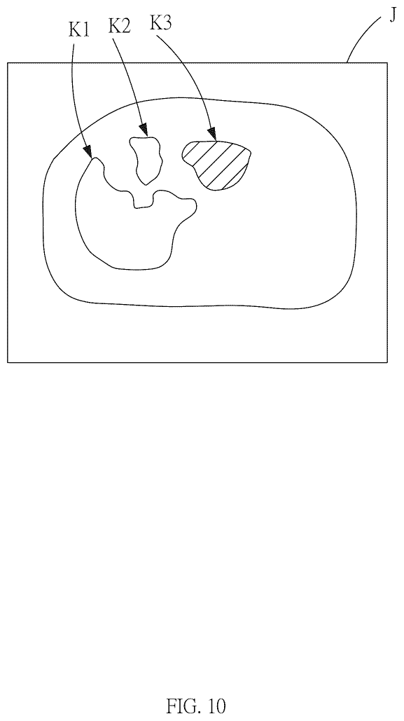

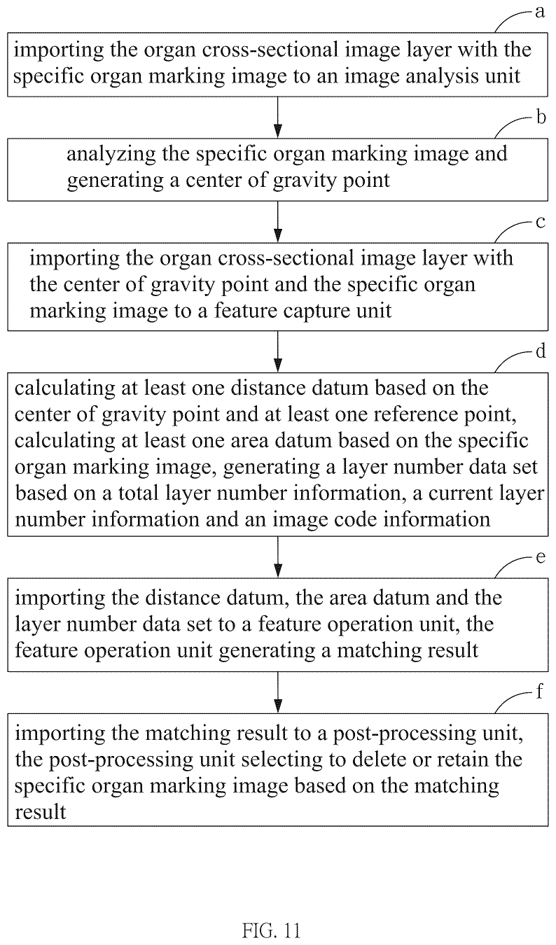

The post-processing unit 204 selects to delete or retain the specific organ marking image K according to the matching result T, wherein the post-processing unit 204 is an image processing unit (IPU) that can be used to receive, process and output image data, and can perform various image processing operations, such as image enhancement, noise reduction, edge detection and color adjustment. The post-processing unit 204 can be an independent module, or integrated to a processing unit (such as CPU, GPU, NPU), or a dedicated image processing chip (such as ISP, Image Signal Processor). To further illustrate, the matching result T is imported to the post-processing unit 204. When the matching result T is false positive data, the specific organ marking image K is deleted. On the contrary, if the matching result T is correct data, the specific organ marking image K is retained. Please refer to FIG. 10. In this embodiment, the specific organ marking image K3 (area with inclined lines) will be deleted from the organ cross-sectional image layer J.

Thus, in medical image analysis, a very large number of samples (organ cross-sectional image layers) are not required, because a large number of samples are not easy to obtain and there will be problems with personal data. In addition, even if a large number of samples are obtained, training costs and computing costs will be high. Therefore, the invention is a feature capturing method designed based on organ anatomy to obtain the distance data S, the area data R and the layer number data set Q of an organ region, which are used as a classification basis for judging whether the organ region is positive or false positive, thereby reducing a false positive rate and improving an accuracy rate. An advantage of this method is that it does not require a very large amount of data and costs.

Please refer to FIG. 11. The invention is a three-dimensional organ medical image feature capturing method for reducing false positives, the three-dimensional organ medical image feature capturing method for reducing false positives obtaining a plurality of organ cross-sectional image layers of a same type of three-dimensional organs by a magnetic resonance imaging (MRI), generating at least one specific organ marking image by an AI analysis system for the organ cross-sectional image layer of each of the organs, comprising:
a. importing the organ cross-sectional image layer with the specific organ marking image to an image analysis unit;
b. analyzing the specific organ marking image and generating a center of gravity point;
c. importing the organ cross-sectional image layer with the center of gravity point and the specific organ marking image to a feature capture unit;
d. calculating at least one distance datum based on the center of gravity point and at least one reference point, calculating at least one area datum based on the specific organ marking image, generating a layer number data set based on a total layer number information, a current layer number information and an image code information;
e. importing the distance datum, the area datum and the layer number data set to a feature operation unit, the feature operation unit generating a matching result; and
f. importing the matching result to a post-processing unit, the post-processing unit selecting to delete or retain the specific organ marking image based on the matching result.

The above-mentioned embodiments are merely exemplification to illustrate the principles and efficacies of the invention, and are not used to limit the invention. Any person having ordinary skill in the art can modify the above-mentioned embodiments without departing from the spirit and scope of the invention. Therefore, the scope of protection of the rights of the invention should be as listed in the appended claims hereinafter.

What is claimed is:

1. A three-dimensional organ medical image feature capturing system for reducing false positives, a plurality of organ cross-sectional image layers of a same type of three-dimensional organs being obtained by a magnetic resonance imaging (MRI), at least one specific organ marking image being generated by an AI analysis system for the organ cross-sectional image layer of each of the organs, the system comprising:
an image analysis unit for importing the organ cross-sectional image layer with the specific organ marking image, the image analysis unit generating a center of gravity point for the specific organ marking image;
a feature capture unit for importing the organ cross-sectional image layer with the center of gravity point and the specific organ marking image, the feature capture unit calculating at least one distance datum based on the center of gravity point and at least one reference point, calculating at least one area datum based on the specific organ marking image, generating a layer number data set based on a total layer number information, a current layer number information and an image code information;
a feature operation unit for importing the distance datum, the area datum and the layer number data set and generating a matching result; and
a post-processing unit, the post-processing unit selecting to delete or retain the specific organ marking image based on the matching result.

2. The three-dimensional organ medical image feature capturing system for reducing false positives as claimed in claim 1, further comprising a storage unit, the storage unit storing the reference point and being used for importing the reference point to the feature capture unit.

3. The three-dimensional organ medical image feature capturing system for reducing false positives as claimed in claim 1, wherein the organ cross-sectional image layer comprises the total layer number information and the current layer number information.

4. The three-dimensional organ medical image feature capturing system for reducing false positives as claimed in claim 2, wherein before the system is used, the reference point must be established and stored in the storage unit, the organ cross-sectional image layers are used to mark reference truth regions, the image analysis unit is then used to generate reference center of gravity points of the reference truth regions, and the reference point is obtained by performing an average calculation on the reference center of gravity points.

5. A three-dimensional organ medical image feature capturing method for reducing false positives, obtaining a plurality of organ cross-sectional image layers of a same type of three-dimensional organs by a magnetic resonance imaging (MRI), generating at least one specific organ marking image by an AI analysis system for the organ cross-sectional image layer of each of the organs, comprising:
a. importing the organ cross-sectional image layer with the specific organ marking image to an image analysis unit;
b. analyzing the specific organ marking image and generating a center of gravity point;
c. importing the organ cross-sectional image layer with the center of gravity point and the specific organ marking image to a feature capture unit;
d. calculating at least one distance datum based on the center of gravity point and at least one reference point, calculating at least one area datum based on the specific organ marking image, generating a layer number data set based on a total layer number information, a current layer number information and an image code information;
e. importing the distance datum, the area datum and the layer number data set to a feature operation unit, the feature operation unit generating a matching result; and
f. importing the matching result to a post-processing unit, the post-processing unit selecting to delete or retain the specific organ marking image based on the matching result.

6. The three-dimensional organ medical image feature capturing method for reducing false positives as claimed in claim 5, wherein the reference point is imported to the feature capture unit from a storage unit.

7. The three-dimensional organ medical image feature capturing method for reducing false positives as claimed in claim 5, wherein the organ cross-sectional image layer comprises the total layer number information and the current layer number information.

8. The three-dimensional organ medical image feature capturing method for reducing false positives as claimed in claim 5, wherein before the system is used, the reference point must be established and stored in the storage unit, the organ cross-sectional image layers are used to mark reference truth regions, the image analysis unit is then used to generate reference center of gravity points of the reference truth regions, and the reference point is obtained by performing an average calculation on the reference center of gravity points.
